(19) United States
(12) Patent Application Publication
(10) Pub. No.: US 2025/0221915 A1
(43) Pub. Date: Jul. 10, 2025

(54) IMPROVEMENT OF THE WATER RESISTANCE OF COSMETIC COMPOSITIONS COMPRISING UV FILTERS

(71) Applicant: DSM IP Assets B.V., Maastricht (NL)

(72) Inventors: Anne JANSSEN, Kaiseraugst (CH); Karina RADOMSKY, Kaiseraugst (CH); Christophe VILAIN, Kaiseraugst (CH); Christine MENDROK-EDINGER, Kaiseraugst (CH)

(21) Appl. No.: 18/853,279

(22) PCT Filed: Apr. 3, 2023
(86) PCT No.: PCT/EP2023/058643

§ 371 Date: Oct. 1, 2024

(30) Foreign Application Priority Data
Apr. 4, 2022 (EP) 22166557.3

(51) Int. Cl. A61K 8/73; A61K 8/29; A61K 8/31; A61K 8/35; A61K 8/37; A61K 8/40; A61K 8/49; A61K 8/891; A61Q 17/04

(52) U.S. Cl. CPC A61K 8/732; A61K 8/29; A61K 8/31; A61K 8/35; A61K 8/37; A61K 8/40; A61K 8/4946; A61K 8/496; A61K 8/4966; A61K 8/731; A61K 8/891; A61Q 17/04

(57) ABSTRACT

The present invention relates to cosmetic composition comprising UV-filter(s), an ester of a fatty acid and dextrin and a mixture of branched and linear saturated C15-C19, wherein said mixture comprised primarily branched saturated C15-C19 and a specific cellulose derivative. This cosmetic composition shows a significantly increased water resistance.

### TECHNICAL FIELD

The present invention relates to the field of cosmetic compositions which protect from UV light.

### BACKGROUND OF THE INVENTION

The trend away from elegant pallor towards “healthy, sporty brown skin” has been unbroken for years. In order to achieve this, people expose their skin to the sun's rays, as this causes pigment formation in the sense of melanin formation. However, the ultraviolet radiation of sunlight also has a damaging effect on the skin. In addition to acute damage (sunburn), long-term damage such as an increased risk of skin cancer occurs if the skin is excessively exposed to light from the UV(B) range (wavelength: 280-315 nm). Excessive exposure to UV(B) and UV(A) radiation (wavelength: 315-400 nm) also weakens the elastin and collagen fibers of the connective tissue. This leads to numerous phototoxic and photoallergic reactions and results in premature skin ageing.

To protect the skin, a series of light protection filter substances have therefore been developed which can be used in cosmetic preparations.

The capacity of a cosmetic composition to protect skin from UV radiation is typically given by the Sun Protection Factor (SPF).

Exposure of a sun protecting cosmetic composition to water, such as typically occurring when bathing, results typically in a significant removal of the UV-filter from the skin, which leads to a decrease of protection capacity against the UV light. This can be shown by a significant decrease of the composition's SPF.

### SUMMARY OF THE INVENTION

Therefore, the problem to be solved by the present invention is to increase the water resistance of a cosmetic composition comprising at least one UV-filter.

Surprisingly, it has been found that the cosmetic composition according to claim 1 allows to solve this problem.

Particularly, a significant increase in the water resistance of said cosmetic composition comprising a UV filter can be obtained when a specific cellulose in combination with a mixture of branched and linear saturated C15-C19 alkanes, and an ester of a fatty acid and dextrin is added to a cosmetic composition comprising a UV filter.

Particularly, it has been found that the triple combination (specific cellulose in combination/mixture of branched and linear saturated C15-C19 alkanes/ester of a fatty acid and dextrin) is significantly higher than the double combinations (specific cellulose in combination/mixture of branched and linear saturated C15-C19 alkanes) (mixture of branched and linear saturated C15-C19 alkanes/ester of a fatty acid and dextrin).

Further aspects of the invention are subject of further independent claims. Particularly preferred embodiments are subject of dependent claims.

### DETAILED DESCRIPTION OF THE INVENTION

In a first aspect the present invention relates to a cosmetic composition comprising
a UV-filter
a mixture of branched and linear saturated C15-C19 alkanes; and
an ester of a fatty acid and dextrin; and
a cellulose which is a C₁₋₆-alkylcellulose or a hydroxy-C₂₋₆-alkyl (C₁₋₆-alkyl)cellulose or carboxy C₁₋₆-alkyl (C₁₋₆-alkyl)cellulose
wherein the amount of branched saturated C15-C19 alkane in said mixture of branched and linear saturated C15-C19 alkanes is more than 80% by weight, preferably more that 90% by weight, most preferred more than 92% by weight.

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

Analogously, a “C_{x-y}-alkylene” group is an alkylene group comprising x to y carbon atoms, i.e., for example, a C₁₋₃-alkylene group is an alkylene group comprising 1 to 3 carbon atoms. The alkylene group can be linear or branched. For example, —CH₂—CH₂—CH₂— and —CH(CH₃)—CH₂— and —C(CH₂—CH₃)— and —C(CH₃)₂— are all considered as a C₃-alkylene group.

A “C_{x-y}-alkylcellulose” as used in the present document is a cellulose with carries an alkylated C_{x-y}-alkyl groups, in other words the cellulose comprises instead of —OH groups some —O—C_{x-y}-alkyl group at the cellulose. For example, a C₁-alkylcellulose is methylcellulose and C₂-alkylcellulose is ethylcellulose.

The term “hydroxy-C_{x-y}-alkyl (C_{x-y}-alkyl)cellulose” is to be understood as to describe hydroxy-C_{x-y}-alkyl cellulose as well as by hydroxy-C_{x-y}-alkyl C_{x-y}-alkylcellulose.

A “hydroxy-C_{x-y}-alkyl cellulose” is a cellulose of which certain —OH groups are replaced by —O—C_{x-y}-alkylene-OH groups. For example, a hydroxy-C₃-alkyl cellulose is hydroxypropyl cellulose.

A “hydroxy-C_{x-y}-alkyl C_{x-y}-alkylcellulose” is a cellulose of which certain —OH groups are replaced by —O—C_{x-y}-alkylene-OH groups as well as by —O—C_{x-y}-alkyl groups. For example, hydroxy-C₂-alkyl C₁-alkyl cellulose is hydroxyethyl methylcellulose.

The term “carboxy C_{x-y}-alkyl (C_{x-y}-alkyl)cellulose” is analogously to be understood as to describe as well carboxy-C_{x-y}-alkyl cellulose as carboxy C_{x-y}-alkyl C_{x-y}alkylcellulose.

A “carboxy-C_{x-y}-alkyl cellulose” is a cellulose of which certain —OH groups are replaced by —O—C_{x-y}-alkylene-COOH or —O—C_{x-y}-alkylene-COO⁻ groups. For example, carboxy-C1-alkyl cellulose is carboxymethyl cellulose.

A “carboxy C_{x-y}-alkyl C_{x-y}alkylcellulose” is a cellulose of which certain —OH groups are replaced by —O—C_{x-y}-alkyl groups as well as by —O—C_{x-y}-alkylene-COOH or —O—C_{x-y}-alkylene-COO⁻ groups. For example, carboxymethyl ethylcellulose or sodium carboxymethyl ethylcellulose are both considered to be carboxy C₁-alkyl C₂-alkylcellulose.

In case identical labels for symbols or groups are present in several formulae, in the present document, the definition of said group or symbol made in the context of one specific formula applies also to other formulae which comprises the same said label.

The term “UV filter” in the present document stands for a substance that absorbs ultraviolet light (=UV light), i.e. electromagnetic radiation of the wavelength between 280 and 400 nm. UV(A) filters are UV filters that absorb UV(A) light, i.e. electromagnetic radiation of the wavelength between 315 and 400 nm. UV(B) filters are UV filters that absorb UV(B) light, i.e. electromagnetic radiation of the wavelength between 280 and 315 nm.

A liquid organic UV filter is liquid at ambient temperature (i.e. 25° C.).

A solid organic UV filter is solid at ambient temperature (i.e. 25° C.).

A “mixture of branched and linear saturated C15-C19 alkanes” in the present document means that said mixture comprises different alkanes each of them only having 15, 16, 17, 18 or 19 carbon atoms but does not comprise any alkanes having less carbons. Therefore, such a mixture does not contain for example dodecane or isododecane. Said mixture comprises both branched and linear C15-C19 alkanes.

### Mixture of Branched and Linear Saturated C15-C19 Alkanes

The cosmetic composition comprises a mixture of branched and linear saturated C15-C19 alkanes.

Particular suitable mixtures of C15-C19 alkanes are particularly the ones disclosed in WO 2016/185046, WO 2017/046177, WO 2018/109353 A1 and WO 2018/109354 A1 and WO 2018/172228 A1.

Preferably, the mixture of branched and linear saturated C15-C19 alkanes has a content of carbon of biological origin being greater or equal to 90% with respect of the total weight of the mixture of branched and linear saturated C15-C19 alkanes. The biological origin of chemicals is very advantageous as such material has a high degree of sustainability. High sustainable products or compositions are highly demanded in the market.

The determination of the content of biomaterial or content of biocarbon is given pursuant to standards ASTM D 6866-12, method B (ASTM D 6866-06) and ASTM D 7026 (ASTM D 7 026-04). Standard ASTM D 6866 concerns “Determining the Biobased Content of Natural Range Materials Using Radiocarbon and Isotope Ratio Mass Spectrometry Analysis”, while standard ASTM D 7 026 concerns “Sampling and Reporting of Results for Determination of Biobased Content of Materials via Carbon Isotope Analysis”. The second standard mentions the first in its first paragraph. The first standard describes a test of measurement of the ratio ¹⁴C/¹²C of a sample and compares it with the ratio ¹⁴C/¹²C of a sample renewable reference of origin 100%, to give a relative percentage of C of origin renewable in the sample. The standard is based on the same concepts that the dating with ¹⁴C.

It is further preferred that the composition has no or a very small amount (less than 100 ppm, particularly less than 30 ppm) of aromatic hydrocarbons with respect to the total weight of the mixture of branched and linear saturated C15-C19 alkanes.

The mixture of branched and linear saturated C15-C19 alkanes is particularly produced by catalytic hydrogenation of hydrocarbon biomass feedstock, such as described in detail in WO 2016/185046, particular the one disclosed as example 3 of WO 2016/185046.

It is preferred that the amount of linear saturated C15-C19 alkanes in said mixture of branched and linear saturated C15-C19 alkanes is less than 10% by weight, preferably less than 8% by weight, most preferred more than 5% by weight.

It is further preferred that the amount of C15 is less than 3%, particularly less than 1%, preferably less than 0.05%, by weight in respect to the weight of said mixture of branched and linear saturated C15-C19 alkanes.

It is preferred that the mixture of branched and linear saturated C15-C19 alkanes is a mixture of branched and linear saturated C16-C19 alkanes.

It is further preferred that amount of branched saturated C16-C18 alkane is more than 90% by weight, preferably more than 95% by weight in respect to the weight of said mixture of branched and linear saturated C15-C19 alkanes.

It is further preferred that the amount of C15 alkanes is less than 5%, particularly less than 2%, by weight in respect to the weight of said mixture of branched and linear saturated C15-C19 alkanes.

It is further preferred that amount of branched saturated C17-C18 alkane is more than 85% by weight, preferably more than 92% by weight in respect to the weight of said mixture of branched and linear saturated C15-C19 alkanes.

It is further preferred that the amount of C17 alkanes is more between 15 and 20% by weight in respect to the weight of said mixture of branched and linear saturated C15-C19 alkanes.

It is further preferred that amount of branched saturated C18 alkane is more than 50% by weight, preferably more than 60% by weight, even more preferably more than 70% by weight, relative to the weight of said mixture of branched and linear saturated C15-C19 alkanes.

It is further preferred that the amount of C18 alkanes is particularly between 70 and 75% by weight in respect to the weight of said mixture of branched and linear saturated C15-C19 alkanes.

In other words, the mixture of branched and linear saturated C15-C19 alkanes consist preferably mainly of C18 alkane(s), most preferably mainly of branched C18 alkane(s).

As the composition comprises a mixture of branched and linear saturated C15-C19 alkanes, said composition does not comprise any lower alkanes, i.e. it does particularly not comprise any C12 alkanes and particularly does not comprise any C12 or C13 or C14 alkanes.

It is further preferred that the mixture of C15-C19 alkanes has at 20° C., a viscosity of 3-15 mPa·s, particularly between 6 and 12 mPa·s.

It is further preferred that the mixture of C15-C19 alkanes has at 20° C. a refractive index of between 1.40 and 1.48, particularly of between 1.42 and 1.45, most preferably between 1.43 and 1.44.

It is further preferred that the mixture of C15-C19 alkanes is the mixtures of C15-C19 alkanes as commercialized as EMOGREEN™ L19 by SEPPIC.

The amount of the mixture of branched and linear saturated C15-C19 alkanes is in the range of between 0.5 and 12.0% by weight, particularly between 1.0 and 10.0% by weight, more preferably between 1.5 and 8.0% by weight, preferably between 2.0 and 6.0% by weight, based on the weight of the hair care.

### Ester of a Fatty Acid and Dextrin

The composition further comprises an ester of a fatty acid and dextrin.

Dextrin is an oligomer of D-glucose. Its structure can be represented simplified by the following structure

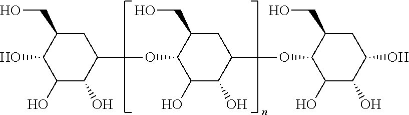

Dextrins have different average degrees of glycopolymerization which leads to different molecular weights.

In the present invention, the dextrin of said ester of a fatty acid and dextrin has preferably an average degree of glycopolymerization of between 3 and 20, particularly between 8 and 16.

It is preferred that the fatty acid of said ester of a fatty acid and dextrin is a C14-C18 fatty acid, particularly a linear C14-C18 fatty acid, most preferably palmitic acid.

As particular suitable ester of a fatty acid and dextrin is a dextrin palmitate as commercialized as Rheopearl® KL2 by Chiba Flour Milling.

Dextrin has several hydroxyl groups which can be esterified.

It is preferred that said ester of a fatty acid and dextrin has an average number of esterified hydroxyl groups of more than 2.5, preferably between 2.5 and 3.5, more preferably between 2.5 and 3.4, most preferably between 2.5 and 3.2, per glucose unit.

In one embodiment said ester of a fatty acid and dextrin has an average number of esterified hydroxyl groups of more than 2.5, preferably between 2.7 and 3.5, more preferably between 28 and 3.4, most preferably between 2.8 and 3.2, per glucose unit.

In another embodiment said ester of a fatty acid and dextrin has an average number of esterified hydroxyl groups of more than 3, preferably between 3.05 and 3.5, more preferably between 3.1 and 3.4, most preferably between 3.1 and 3.2, per glucose unit.

In other words, preferably essentially all of the hydroxyl groups of the dextrin are esterified.

It is further preferred that said ester of a fatty acid and dextrin has a molecular weight Mₙ of between 8,000 and 16,000 Da, preferably between 9,000 and 13,000 Da, more preferably between 10,000 and 11,500 Da.

The molecular weight Mn is determined in Dalton (Da) particularly by SEC/GPC using polystyrene as standard.

Both fatty acid and dextrin have biological origin. The biological origin of chemicals is very advantageous as such material or products thereof have a high degree of sustainability. High sustainable products or compositions are highly demanded in the market.

The amount of the ester of a fatty acid and dextrin is preferably in the range of between 0.1 and 15% by weight, particularly between 0.3 and 8.0% by weight, particularly between 0.6 and 7.0% by weight, more preferably between 1.0 and 6.0% by weight, particularly between 1.5 and 5.0% by weight, based on the weight of the cosmetic composition.

In the said composition, the ratio of the weight of said ester of a fatty acid and dextrin to the weight of said mixture of branched and linear saturated C15-C19 alkanes is preferably less than 1, preferably in the range of 0.5 to 0.8, most preferred in the range of 0.55-0.70.

In other words, the composition comprises preferably more, by weight, of the C15-C19 alkanes than of the ester of a fatty acid and dextrin.

### UV Filter

The composition further comprises a UV filter. The UV filter is either an inorganic or an organic UV filter, preferably a liquid organic UV filter or a solid organic UV filter.

Suitable liquid organic UV-filter absorb light in the UVB and/or UVA range and are liquid at ambient temperature (i.e. 25° C.). Such liquid UV-filter are well known to a person in the art and encompass in particular cinnamates such as e.g. octyl methoxycinnamate (PARSOL® MCX) and isoamyl methoxycinnamate (Neo Heliopan® E 1000), salicylates such as e.g. homosalate (3,3,5 trimethylcyclohexyl 2-hydroxybenzoate, PARSOL® HMS) and ethylhexyl salicylate (also known as ethylhexyl salicylate, 2 ethylhexyl 2-hydroxybenzoate, PARSOL® EHS), acrylates such as e.g. octocrylene (2 ethylhexyl 2-cyano-3,3-diphenylacrylate, PARSOL® 340) and ethyl 2-cyano-3,3 diphenylacrylate, esters of benzalmalonic acid such as in particular dialkyl benzalmalonates such as e.g. di (2-ethylhexyl) 4-methoxy-benzalmalonate and polysilicone 15 (PARSOL® SLX), dialkylester of naphthalates such as e.g. diethylhexyl 2,6-naphthalate (Corapan® TQ), syringylidene malonates such as e.g. diethylhexyl syringylidene malonate (Oxynex® ST liquid) as well as benzotriazolyl dodecyl p-cresol (Tinoguard® TL) as well as benzophenone-3 and drometrizole trisiloxane.

It has been observed that polysilicone based organic UV filters (such as polysilicon 15) have a less pronounced reduction in material transfer. Hence, it is preferred that the Cosmetic composition does not comprise a polysilicon based organic UV filter.

Particular advantageous liquid organic UV-filter are octyl methoxycinnamate, homosalate, ethylhexyl salicylate, octocrylene, diethylhexyl 2,6-naphthalate, diethylhexyl syringylidene malonate, benzotriazolyl dodecyl p-cresol, benzo-phenone-3, drometrizole trisiloxane as well as mixtures thereof.

In a preferred embodiment, the liquid UV filter is a liquid UV(B) filter which is selected from the group consisting of ethylhexyl methoxycinnamate, octocrylene, homosalate, ethylhexyl salicylate, benzophenone-3 and drometrizole trisiloxane.

Suitable solid organic UV-filter absorb light in the UVB and/or UVA range and are solid at ambient temperature (i.e. 25° C.). Particularly suited solid UV-filters are of the group consisting of bis-ethylhexyloxyphenol methoxyphenyl triazine, butyl methoxydibenzoyl methane, methylene bis-benzotriazolyl tetramethylbutylphenol, diethylamino hydroxybenzoyl hexyl benzoate, ethylhexyl triazone, diethylhexyl butamido triazone, 4-methylbenzylidene camphor and 1,4-di(benzoxazol-2′-yl)benzene.

A preferred solid organic UV(A) filter is a UV(A) filter which is selected from the group consisting of bis-ethylhexyloxyphenol methoxyphenyl triazine, butyl methoxydibenzoyl methane, methylene bis-benzotriazolyl tetramethylbutylphenol and diethylamino hydroxybenzoyl hexyl benzoate.

A preferred solid organic UV(B) filter is a UV(B) filter which is selected from the group consisting of ethylhexyl triazone (=Uvinul T150), diethylhexyl butamido triazone (=Uvasorb HEB), and 4-methylbenzylidene camphor (=Parsol 5000).

The total amount of organic UV filter (s) depends strongly on the targeted UV protection.

It is preferred that the amount of a solid organic UV filter, particular of solid organic UV(A) filter, is selected in the range of 0.1 to about 6 wt.-%, preferable in the range of 0.5 to 5 wt.-%, most preferably in the range of 1 to 4 wt.-%.

It is further preferred that amount of a solid organic UV filter, particular of solid organic UV(B) filter, is selected in the range of 0.1 to about 6 wt.-%, preferable in the range of 0.5 to 5 wt.-%, most preferably in the range of 1 to 4 wt.-%.

It is even further preferred that amount of a liquid organic UV filter, particular of liquid organic UV(B) filter, is selected in the range of 0.1 to about 10 wt.-%, preferable in the range of 0.5 to 8 wt.-%, most preferably in the range of 1 to 6 wt.-%.

In one of the embodiments, the cosmetic composition comprises at least two UV filters of which at least one is a solid or liquid organic UV(A) filter and of which at least one is a solid or liquid organic UV(B) filter.

In said embodiment it is preferred that the composition comprises at least two organic UV filters of which at least one is a solid or liquid organic UV(A) filter and of which at least one is a solid or liquid organic UV(B) filter.

Preferred is a combination of at least one solid organic UV(A) filter and at least one liquid organic UV(B) filter.

Particularly preferred in said embodiment is a combination of at least one solid organic UV(A) filter which is selected from the group
consisting of bis-ethylhexyloxyphenol methoxyphenyl triazine, butyl methoxydibenzoyl methane, methylene bis-benzotriazolyl tetramethylbutylphenol and diethylamino hydroxybenzoyl hexyl benzoate
and
at least one liquid organic UV(B) filter selected from the group consisting of ethylhexyl methoxycinnamate, octocrylene, homosalate, ethylhexyl salicylate and benzophenone-3, drometrizole trisiloxane

The total amount of organic UV filter(s) depends strongly on the targeted UV protection of said composition and is typically in the range of between 1 to 50% by weight, preferably between 5 to 40% by weight, based on the total weight of said composition.

A sun cream with an SPF 15, for example, comprises preferably a total amount of organic UV filter (s) of between 4 to 20% by weight, more preferably between 7 and 15% by weight, based on the total weight of said composition.

A sun cream with an SPF 30, for example, comprises preferably a total amount of organic UV filter (s) of between 10 to 40% by weight, more preferably between 15 and 25% by weight, based on the total weight of said composition.

A sun cream with an SPF 50, for example, comprises preferably a total amount of organic UV filter (s) of between 15 to 50% by weight, more preferably between 20 and 40% by weight, based on the total weight of said composition.

In one of the preferred embodiments, the cosmetic composition comprises the UV-filter of the formula (I)

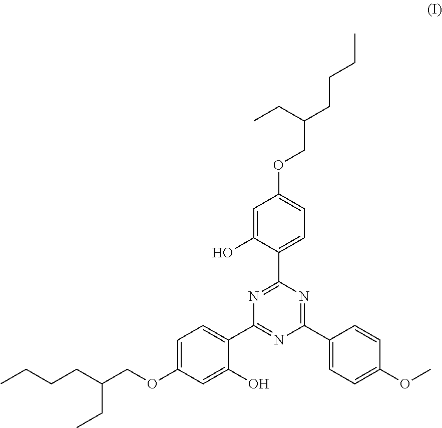 [SMILES: CCCCC(CC)COc1ccc(-c2nc(-c3ccc(OC)cc3)nc(-c3ccc(OCC(CC)CCCC)cc3O)n2)c(O)c1]

The UV-filter of the formula (I) (CAS: [187393-00-6]) is a crystalline solid having a melting point of 83-85° C. and is also known as Bemotrizinol or 2,4-Bis-{[4-(2-ethyl-hexyloxy)-2-hydroxy]-phenyl}-6-(4-methoxyphenyl)-1,3,5-triazine or bis-ethylhexyloxyphenol methoxyphenyl triazine (INCI). It is a broad-spectrum UV absorber, absorbing UV(B) as well as UV(A) light. It has two absorption maxima at 310 and 345 nm and is highly photostable. It is the most effective UV absorber available measured by SPF, based on the maximum concentration permitted by European legislation.

It is commercially available for example under the trademark PARSOL® Shield from DSM Nutritional Products Ltd., Tinosorb® S from BASF and Escalol™ S UV filter from Ashland.

Due to its polarity, it is very soluble in esters, particularly in C12-15 alkyl benzoate, however, is insoluble in water and alkanes.

In another preferred embodiment, the cosmetic composition comprises the UV-filter of the formula (II)

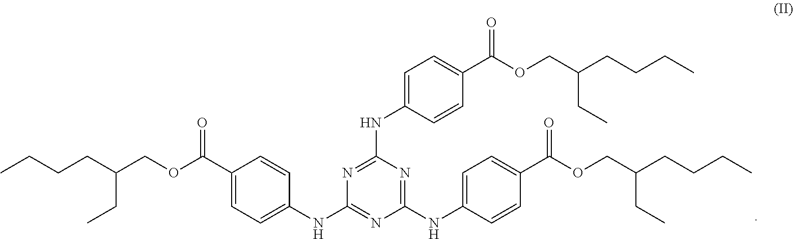 [SMILES: CCCCC(CC)COC(=O)c1ccc(Nc2nc(Nc3ccc(C(=O)OCC(CC)CCCC)cc3)nc(Nc3ccc(C(=O)OCC(CC)CCCC)cc3)n2)cc1]

The UV-filter of the formula (II) (CAS: [88122-99-0]) is a crystalline solid having a melting point of 129° C. and is also known as ethylhexyl triazone (INCI) or as octyltriazone. The UV filter is a highly effective UV(B) filter with an exceptional high absorptivity. It has an absorption maximum of 314 nm. It is commercially available for example under the trademark Uvinul® T 150 from BASF.

Due to its polarity, it is very soluble in esters, particularly in ethyl acetate and ethanol, however, is insoluble in water and alkanes.

In another preferred embodiment, the cosmetic composition comprises the UV-filter of the formula (III)

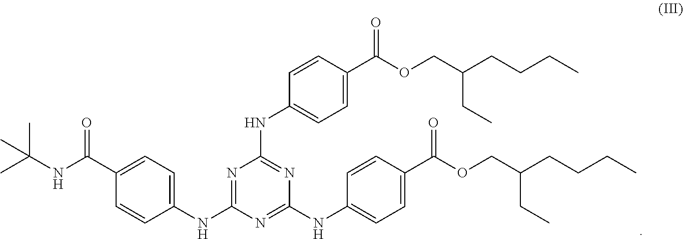 [SMILES: CCCCC(CC)COC(=O)c1ccc(Nc2nc(Nc3ccc(C(=O)NC(C)(C)C)cc3)nc(Nc3ccc(C(=O)OCC(CC)CCCC)cc3)n2)cc1]

The UV-filter of the formula (III) (CAS: [154702-15-5]) is a light yellow crystalline solid with a melting point of 52-55° C. and is also known as Iscotrizinol or diethylhexyl butamido triazone (INCI). The UV filter is a highly effective UV(A) and UV(B) filter with an exceptional high absorptivity and is very highly photostable.

It has an absorption maximum of about 310 nm. It is commercially available for example under the trademark UVASORB® HEB from 3V Sigma.

Due to its polarity, it is very soluble in esters, and alcohols, particularly in ethanol, however, is insoluble in water and alkanes.

In another preferred embodiment, the cosmetic composition comprises the UV-filter of the formula (IV)

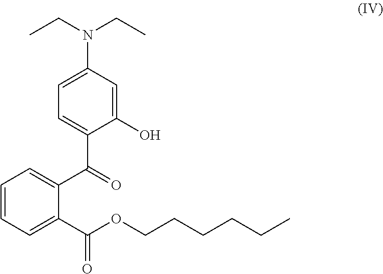 [SMILES: CCCCCCOC(=O)c1ccccc1C(=O)c1ccc(N(CC)CC)cc1O]

The UV-filter of the formula (IV) (CAS: [302776-68-7]) is a crystalline solid having a melting point of 54° C. and is also known as diethylamino hydroxybenzoyl hexyl benzoate (INCI). The UV filter is a highly effective UV(A) filter with a relative broad absorption with an absorption maximum at 354 nm. It is commercially available for example under the trademark Uvinul® A Plus from BASF.

Due to its polarity, it is very soluble in esters, particularly in ethyl hexyl-benzoate and ethanol, however, is insoluble in water and alkanes.

In another preferred embodiment, the cosmetic composition comprises the UV-filter of the formula (V)

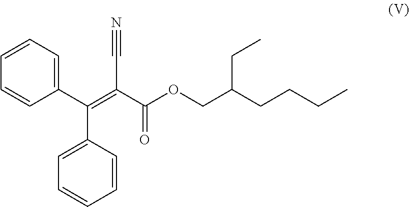 [SMILES: CCCCC(CC)COC(=O)C(C#N)=C(c1ccccc1)c1ccccc1]

The UV-filter of the formula (V) (CAS: [6197-30-4]) is a viscous, oily liquid with a melting point of 14° C. that is clear and colorless. It is also known as 2-ethylhexyl 2-cyano-3,3-diphenyl-2-propenoate and as Octocrylene (INCI). The extended conjugation of the acrylate portion of the molecule absorbs UV(B) rays with wavelengths from 280 to 320 nm protecting the skin from direct DNA damage. It has an absorption maximum at 310 nm. The UV-filter of the formula (V) has an excellent photostability and is an excellent solubilizer for solid, crystalline UV filters. It is commercially available for example under the trademark PARSOL®340 from DSM Nutritional Products Ltd. or Uvinul® N 539T from BASF. Most preferred is the product PARSOL® 340 from DSM Nutritional Products Ltd.

Due to its polarity, Octocrylene is has a good solubility in propylene carbonate and dipropylene glycol, however, is insoluble in water and alkanes.

In another preferred embodiment, the cosmetic composition comprises the UV-filter of the formula (VI)

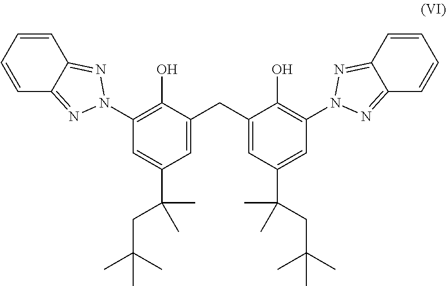 [SMILES: CC(C)(C)CC(C)(C)c1cc(Cc2cc(C(C)(C)CC(C)(C)C)cc(-n3nc4ccccc4n3)c2O)c(O)c(-n2nc3ccccc3n2)c1]

The UV-filter of the formula (VI) (CAS: [103597-45-1]) is a solid with a melting point of 197-199° C. It is also known as Bisoctrizole or as methylene bis-benzotriazolyl tetramethylbutylphenol (INCI).

Bisoctrizole is a broad-spectrum ultraviolet radiation filter, absorbing UV(B) as well as UV(A) rays and has an excellent photostability. It has an absorption maximum at 308 nm and 349 nm. However, next to absorption of UV light, it also reflects and scatters UV light. Therefore, Bisoctrizole is a hybrid UV absorber, an organic UV filter produced in microfine organic particles (<200 nm). Where other organic UV filters need to be dissolved in either the oil or water phase, bisoctrizole dissolves poorly in both and is applied as invisible particle.

Bisoctrizole is mainly available as micronized powder in a 50% aqueous suspension which is stabilized by the surfactant decyl glucoside for example under the trademark PARSOL® Max from DSM Nutritional Products Ltd. or Tinosorb® M from BASF or as Eversorb M from Everlight Chemical or as Milestab 360. Most preferred is the product PARSOL® Max from DSM Nutritional Products Ltd.

Bisoctrizole has an extreme low solubility in most solvents or cosmetic oils and is almost insoluble, respectively very insoluble, particularly in water and alkanes.

In another preferred embodiment, the cosmetic composition comprises the UV-filter of the formula (VII)

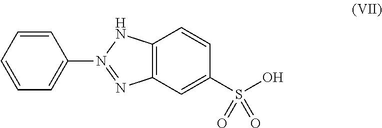

The UV-filter of the formula (VII) (CAS: [27503-81-7]) is a solid with a melting point of 1>300° C. It is also known as phenylbenzimidazole sulfonic acid (INCI name) and as Ensulizole.

Ensulizole is a strong UV(B) light filter, which absorbs particularly light in the range between 280-340 nm, and has an absorption maximum at 302 nm.

Ensulizole is a UV filter which is broadly used in sun care products. Due to its water solubility, Ensulizole is commonly used in products formulated to feel light and less oily. Due to the low absorption properties of UV(A) light, it is typically combined with other UV filters, particularly (UV(A) filters, particularly with avobenzone.

Ensulizole is commercialized under the trademark EUSOLEX® 232 by Merck KGaA and particularly under the trademark PARSOL® HS by DSM Nutritional Products Ltd.

In another preferred embodiment, the cosmetic composition comprises the UV-filter of the formula (VIII)

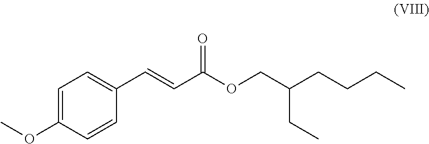 [SMILES: CCCCC(CC)COC(=O)/C=C/c1ccc(OC)cc1]

The UV-filter of the formula (VIII) (CAS: [5466-77-3]) is a clear, yellowish liquid with a boiling point of about 199° C., which is not soluble in water and soluble in fats, oils and alcohol. It is also known as Ethylhexyl Methoxycinnamate (INCI name) and as Octinoxate.

Ethylhexyl Methoxycinnamate is a strong UV(B) light filter which has an absorption maximum at 310 nm.

Ethylhexyl Methoxycinnamate is commercialized under the trademark EUSOLEX® 2292 by Merck KGaA, Uvinul® MC80 by BASF and particularly under the trademark PARSOL® MCX by DSM Nutritional Products Ltd.

In another preferred embodiment, the cosmetic composition comprises the UV-filter Polysilicone-15 of the formula (IX):

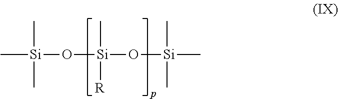

whereas p is approximately 60; and
R is

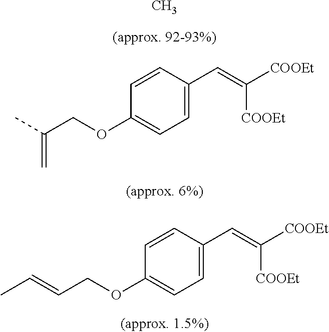

The UV-filter of the formula (IX) (CAS: [207574-74-1]) is a colorless to pale yellow viscous liquid which is not soluble in water but soluble in organic solvents of medium polarity and in typical cosmetic oils. It is also known as Dimethicodiethylbenzalmalonate.

Polysilicone-15 a UV(B) light filter which has an absorption maximum of about at 310 nm.

Polysilicone-15 is commercially available under the trademark PARSOL® SLX from DSM Nutritional Products Ltd.

In another preferred embodiment, the cosmetic composition comprises the inorganic UV-filter which is double coated TiO₂, preferably a rutile type TiO₂, having an inner silica and an outer organic coating, typically dimethicone, and is a UV(B) light filter.

The preferred UV-Filter of this class is commercially available under the trademark PARSOL® TX from DSM Nutritional Products Ltd.

In another preferred embodiment, the cosmetic composition comprises the UV-filter of the formula (X)

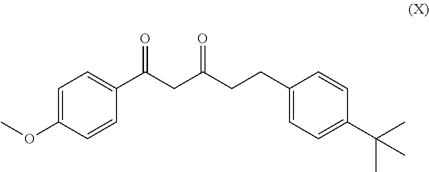 [SMILES: COc1ccc(C(=O)CC(=O)CCc2ccc(C(C)(C)C)cc2)cc1]

The UV-filter of the formula (X) (CAS: [70356-09-1]) is a whitish to yellowish crystalline powder and is insoluble in water and soluble in isopropanol, dimethyl sulfoxide, decyl oleate, capric acid/caprylic, triglycerides and other oils.

The UV-filter of formula (X) is known as 1-(4-Methoxyphenyl)-3-(4-tert-butylphenyl)propane-1,3-dione or as butyl methoxydibenzoylmethane (INCI name) and as Avobenzone.

Avobenzone is an efficient UV(A) light filter which absorbs particularly light in the range between 315 and 380 nm, and has an absorption maximum at 357 nm.

Avobenzone is commercialized under the trademark ESCALOL 517 by Ashland, EUSOLEX® 9020 by Merck KGaA, NEO HELIOPAN 357 by Symrise and particularly under the trademark PARSOL® 1789 by DSM Nutritional Products Ltd.

In another preferred embodiment, the cosmetic composition comprises the UV-filter of the formula (XI)

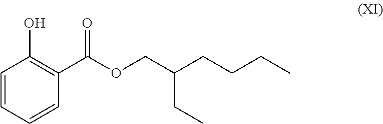 [SMILES: CCCCC(CC)COC(=O)c1ccccc1O]

The UV-filter of the formula (XI) (CAS: [118-60-5]) is a clear colorless liquid and is insoluble in water and other polar solvents and soluble apolar solvents and cosmetic oils. The UV-filter of formula (XI) is known as Ethylhexyl Salicylate (INCI name) or as Octyl salicylate or Octisalate.

Ethylhexyl Salicylate is an effective UV(B) light filter and has an absorption maximum at 305 nm. Although Octisalate is a weaker UV(B) light filter it has superior stability than some other sunscreen active ingredients and does not produce reactive oxygen species when exposed to sunlight. Other UV-filters such as Avobenzone are solubilized by Ethylhexyl Salicylate.

Ethylhexyl Salicylate is commercialized under the trademark ESCALOL 587 by Ashland, EUSOLEX® OS by Merck KGaA, NEO HELIOPAN OS by Symrise and particularly under the trademark PARSOL® EHS 1789 by DSM Nutritional Products Ltd.

Particularly, the UV filter is selected from the group consisting of ethylhexyl triazone, avobenzone, bis-ethylhexyloxyphenol methoxyphenyl triazine, 2-ethylhexyl salicylate, phenylbenzimidazole sulfonic acid, ethylhexyl methoxycinnamate, octocrylene, polysilicone-15, iscotrizinol, titanium dioxide, and methylene bis-benzotriazolyl tetramethylbutylphenol.

Said cosmetic composition has preferably a Sun Protection Factor (SPF) of 10 or higher, preferably of 20 or higher, more preferred of 30 or higher.

### Cellulose

Cellulose is an organic compound with the formula (C₆H₁₀O₅)ₘ, a polysaccharide consisting of a linear chain of very high number (m), typically several hundred to many thousands, of β(1→4) linked D-glucose units.

Cellulose is the starting material for producing C₁₋₆-alkylcellulose or a hydroxy-C₂₋₆-alkyl (C₁₋₆-alkyl)cellulose or carboxy C₁₋₆-alkyl (C₁₋₆-alkyl)cellulose which are of relevance for the present invention.

In one preferred embodiment the cellulose is a C₁₋₆-alkylcellulose, particularly a methylcellulose, an ethylcellulose or a propylcellulose, preferably a methylcellulose or an ethylcellulose. The C₁₋₆-alkylcellulose is typically prepared from cellulose, particularly in alkaline solution, and the respective C₁₋₆-alkylhalogenide.

Preferred is methylcellulose [9004-67-5] which is broadly commercially available, for example as Solterra™ Boost from Dow or as Methocel™ A brand products from Dow, particularly as Methocel™ A4C PCG, Dow More preferred is ethylcellulose [9004-57-3] which is broadly commercially available, particularly as Antaron™ ECo gel from Ashland.

In another preferred embodiment the cellulose is a hydroxy-C₂₋₆-alkyl cellulose, particularly a hydroxyethyl cellulose, hydroxypropyl cellulose or hydroxy-butyl cellulose, preferably a hydroxyethyl cellulose or hydroxypropyl cellulose. The hydroxy-C₂₋₆-alkyl cellulose is typically prepared from cellulose, particularly in alkaline solution, and the respective C₂₋₆-alkyleneoxide.

Preferred is hydroxyethyl cellulose [9004-62-0] which is broadly commercially available, particularly as Natrosol™ Hydroxyethylcellulose (HEC) brand products by Ashland or as ™ brand products by Dow, more particularly as Natrosol™ 250 HHR CS, Ashland, or as ™ HEC QP-15000H Europe PCG Thickener or C™ Hydroxyethyl Cellulose PCG-10 Europe, or ™ QP 4400H Hydroxyethyl Cellulose, Dow.

In another preferred embodiment the cellulose is a hydroxy-C₂₋₆-alkyl C₁₋₆-alkylcellulose, particularly a hydroxyethyl methylcellulose or a hydroxypropyl methylcellulose or a hydroxyethyl ethylcellulose or a hydroxypropyl ethylcellulose, hydroxyethyl propylcellulose or a hydroxypropyl propylcellulose, preferably hydroxyethyl methylcellulose or a hydroxypropyl methylcellulose or a hydroxyethyl ethylcellulose or a hydroxypropyl ethylcellulose.

The hydroxy-C₂₋₆-alkyl C₁₋₆-alkylcellulose is typically prepared from cellulose, particularly in alkaline solution, and the respective C₂₋₆-alkyleneoxide and the respective C₁₋₆-alkylhalogenide.

Preferred is hydroxyethyl methylcellulose [9032-42-2], particularly as commercialized as Structure® CEL 4400 E by Nouryon.

More preferred is hydroxypropyl methylcellulose [9004-65-3], which is broadly commercially available, particularly as Benecel™ K200M from Ashland or as Methocel™ E brand products, Methocel™ F brand products, Methocel™ J brand products and Methocel™ K brand products from Dow, particularly as Methocel™ K15M PCG from Dow.

In another preferred embodiment the cellulose is a carboxy-C₁₋₆-alkyl cellulose, particularly a carboxymethylcellulose, a carboxyethylcellulose or a carboxypropylcellulose, preferably a carboxymethylcellulose, or their salts thereof.

The carboxy-C₁₋₆-alkyl cellulose is typically prepared from cellulose, particularly in alkaline solution, and the respective chloro-C₂₋₇-alkanoic acid, particularly chloroacetic acid.

Preferred is carboxymethylcellulose [Blanose™ sodium carboxymethylcellulose by Ashland or as Cekol® 700 or Cekol® 2000 by CP Kelco.

In another preferred embodiment the cellulose is a carboxyC₁₋₆-alkyl C₁₋₆alkylcellulose, particularly a carboxymethyl methylcellulose, a carboxyethyl methylcellulose or a carboxypropyl methylcellulose, carboxymethyl ethylcellulose, a carboxyethyl ethylcellulose or a carboxypropyl ethylcellulose, carboxymethyl propylcellulose, a carboxyethyl propylcellulose or a carboxypropyl propylcellulose. preferably a carboxymethyl methylcellulose or carboxyethyl methylcellulose, or their salts thereof.

The carboxy C₁₋₆-alkyl C₁₋₆alkylcellulose is typically prepared from cellulose, particularly in alkaline solution, and the respective chloro-C₂₋₇-alkanoic acid, particularly chloroacetic acid, and the respective C₁₋₆-alkylhalogenide.

It is particularly preferred that the cellulose is a selected from the group consisting of methylcellulose, ethylcellulose, propylcellulose, hydroxymethyl cellulose, hydroxyethyl cellulose, hydroxypropyl cellulose, hydroxyethyl methylcellulose, hydroxypropyl methylcellulose, carboxymethyl cellulose, carboxymethyl methylcellulose and carboxymethyl ethylcellulose, preferably an ethylcellulose.

Most preferred is ethylcellulose, particularly Antaron™ ECo gel available from Ashland.

It is preferred that the amount of the cellulose which is a C₁₋₆-alkylcellulose or a hydroxy-C₂₋₆-alkyl (C₁₋₆-alkyl)cellulose or carboxy C₁₋₆-alkyl (C₁₋₆-alkyl) cellulose, is in the range of between 0.01 and 4.0% by weight, particularly between 0.1 and 3.0% by weight, preferably between 0.3 and 2.5% by weight, based on the weight of the cosmetic composition.

### Further Ingredients

The cosmetic composition typically comprises other ingredients which are suitable for the use in cosmetic compositions.

The cosmetic composition comprises preferably water.

The cosmetic compositions may be in the form of a suspension or dispersion in solvents or fatty substances, or alternatively in the form of an emulsion or micro emulsion (in particular of oil-in-water (O/W—) or water-in-oil (W/O—)type, silicone-in-water (Si/W—) or water-in-silicone (W/Si—)type, PIT-emulsion, multiple emulsion (e.g. oil-in-water-in oil (O/W/O—) or water-in-oil-in-water (W/O/W—)type), pickering emulsion, hydrogel, alcoholic gel, lipogel, one- or multiphase solution or vesicular dispersion or other usual forms, which can also be applied by pens, as masks or as sprays.

Preferred cosmetic compositions in all embodiments of the present invention comprise water and are in the form of an emulsion.

The emulsion particularly contains an oily phase and an aqueous phase such as in particular O/W, W/O, Si/W, W/Si, O/W/O, W/O/W multiple or a pickering emulsions.

The total amount of the oily phase present in such emulsions is preferably at least 10 wt.-%, such as in the range from 10 to 60 wt.-%, preferably in the range from 15 to 50 wt.-%, most preferably in the range from 15 to 40 wt.-%, based on the total weight of the cosmetic composition.

The amount of the aqueous phase present in such emulsions is preferably at least 20 wt. %, such as in the range from 40 to 90 wt.-%, preferably in the range from 50 to 85 wt.-%, most preferably in the range from 60 to 85 wt.-%, based on the total weight of the cosmetic composition.

More preferably, the cosmetic compositions are in the form of an oil-in-water (O/W) emulsion comprising an oily phase dispersed in an aqueous phase in the presence of an O/W-respectively Si/W-emulsifier. The preparation of such O/W emulsions is well known to a person skilled in the art.

The compositions in form of O/W emulsions can be provided, for example, in all the formulation forms for O/W emulsions, for example in the form of serum, milk or cream, and they are prepared according to the usual methods. The compositions are preferably intended for topical application and can in particular constitute a dermatological or cosmetic composition, for example intended for protecting human skin against the adverse effects of UV radiation (antiwrinkle, anti-ageing, moisturizing, sun protection and the like).

The cosmetic composition, furthermore, comprises preferably a moisturizer or skin humectant, particularly glycerol and/or panthenol and/or propandiol, and/or Pentavitin, particularly as commercially available as D-Panthenol 75 L, or Tilamar® PDO with Nøøvista or Pentavitin® from DSM Nutritional Products Ltd.

The cosmetic composition further comprises preferably a sensory enhancer, particularly silica, preferably as commercially available as Valvance® Touch 210 from DSM Nutritional Products Ltd.

The cosmetic composition, furthermore, may contain the usual oily and fatty components which may be chosen from mineral oils and mineral waxes; oils such as triglycerides of caprinic acid and/or caprylic acid or castor oil; oils or waxes and other natural or synthetic oils, in a preferred embodiment esters of fatty acids with alcohols e.g. isopropanol, propyleneglycol, glycerin or esters of fatty alcohols with carbonic acids or fatty acids; alkylbenzoates; and/or silicone oils.

Exemplary fatty substances which can be particularly incorporated in the oil phase of the emulsion, microemulsion, oleo gel, hydrodispersion or lipodispersion are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyris-tate, cetearylisononanoate, isopropylmyristate, isopropylpalmitate, isopropyl-stearate, isopropyloleate, n-butylstearate, n-hexyllaurate, n-decyloleate, isooctyl-stearate, isononylstearate, isononylisononanoate, 2-ethyl hexylpalmitate, 2-ethylhexyllaurate, 2-hexyldecylstearate, 2-octyldodecylpalmitate, stearyl-heptanoate, oleyloleate, oleylerucate, erucyloleate, erucylerucate, tridecyl-stearate, tridecyltrimellitate, as well as synthetic, half-synthetic or natural mixtures of such esters e.g. jojoba oil.

Other fatty components, particularly suitable for hair conditioner, include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in the cosmetic composition are isoeikosane; neopentylglycoldiheptanoate; propylene-glycoldicaprylate/dicaprate; caprylic/capric/diglycerylsuccinate; butyleneglycol caprylat/caprat; C₁₂₋₁₃-alkyllactate; di-C₁₂₋₁₃-alkyltartrate; triisostearin; dipenta-erythrityl hexacaprylat/hexacaprate; propyleneglycolmonoisostearate; tricaprylin; dimethylisosorbid. Especially beneficial is the use of mixtures C₁₂₋₁₅-alkylbenzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexylisostearate and isotridecylisononanoate.

The oily phase of the cosmetic composition can also contain natural vegetable or animal waxes such as bees wax, china wax, bumblebee wax and other waxes of insects as well as shea butter and cocoa butter.

Suitable silicone oils are e.g. dimethylpolysiloxane, diethylpolysiloxane, diphenylpolysiloxane, cyclic siloxanes, poly(methylphenylsiloxanes) as well as amino-, fatty acid-, alcohol-, polyether-, epoxy-, fluor-, glycoside-, and/or alkyl modified silicone compounds which are liquid or solid at room temperature and mixtures thereof. The number average molecular weight of the dimethicones and poly(methylphenylsiloxanes) is preferably in the range of 100 to 150,000 g/mol. Preferred cyclic siloxanes comprise 4- to 8-membered rings which are for example commercially available as cyclomethicones.

In one advantageous embodiment, the cosmetic compositions in addition contain a phosphate ester emulsifier. Among the preferred phosphate ester emulsifier are C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-15 Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier is potassium cetyl phosphate, e.g. commercially available as Amphisol® K at DSM Nutritional Products Ltd Kaiseraugst.

The cosmetic composition further may comprise cosmetic carriers, excipients and diluents as well as additives and active ingredients commonly used in the skin care industry which are suitable for use in the cosmetic compositions are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

Such possible ingredients of the cosmetic composition are particularly enhance the performance and/or consumer acceptability such as preservatives, antioxidants, fatty substances/oils, thickeners, softeners, light-screening agents, moisturizers, fragrances, co-surfactants, fillers, sequestering agents, cationic-, nonionic- or amphoteric polymers or mixtures thereof, acidifying or basifying agents, viscosity modifiers, and natural hair nutrients such as botanicals, fruit extracts, sugar derivatives and/or amino acids or any other ingredients usually formulated into cosmetic compositions. The necessary amounts of the adjuvants and additives can, based on the desired product, easily be chosen by a person skilled in the art in this field and will be illustrated in the examples, without being limited hereto.

The cosmetic compositions preferably have a pH in the range from 3 to 10, preferably a pH in the range from 4 to 8 and most preferably a pH in the range from 4 to 7.5. The pH can easily be adjusted as desired with suitable acids such as e.g. citric acid or bases such as NaOH according to standard methods in the art.

In one of the embodiments, the composition is preferably sulfate-free.

The term “free” as used in the present document, for example in “sulfate-free”, is used to mean that the respective substance is only present at amounts of less than 0.5% by weight, particularly less than 0.1% by weight, more particularly below 0.05% by weight, relative to the weight of the composition. Preferably, “free” means that the respective substance is completely absent in the composition.

The cosmetic composition is preferably a topical composition.

The term “topical” as used herein is understood here to mean external application to keratinous substances, which are in particular the skin, scalp, eyelashes, eyebrows, nails, mucous membranes and hair, preferably the skin.

As the topical compositions are intended for topical application, it is well understood that they comprise a physiologically acceptable medium, i.e. a medium compatible with keratinous substances, such as the skin, mucous membranes, and keratinous fibers. In particular, the physiologically acceptable medium is a cosmetically acceptable carrier.

The term “cosmetically acceptable carrier” refers to all carriers and/or excipients and/or diluents conventionally used in cosmetic compositions such as in particular in sun care products.

Preferably the cosmetic composition is a skin care preparation, decorative preparation, or a functional preparation.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

The cosmetic composition is preferably a skin care composition.

In a most preferred embodiment, the cosmetic composition is a sun care composition. Sun care compositions are light-protective preparations (sun care products), such as sun protection milks, sun protection lotions, sun protection creams, sun protection oils, sun blocks or day care creams with a SPF (sun protection factor). Of particular interest are sun protection creams, sun protection lotions, sun protection milks and sun protection preparations.

The cosmetic composition, particularly the topical sunscreen emulsions according to the invention, have in general a pH in the range of 3 to 10, preferably a pH in the range of 5 to 8 and most preferably a pH in the range of 6 to 7.5. The pH can easily be adjusted as desired with suitable acids such as e.g. citric acid or bases such as sodium hydroxide (e.g. as aqueous solution), Triethanolamine (TEA Care), Tromethamine (Trizma Base) and Aminomethyl Propanol (AMP-Ultra PC 2000) according to standard methods in the art.

The amount of the cosmetic composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

It has been shown that the above cosmetic composition has an improved water resistance. It has been shown that by adding said specific cellulose and said mixture of branched and linear saturated C15-C19 alkanes and said ester of a fatty acid and dextrin as mentioned above to cosmetic compositions comprising a UV-Filter, the water resistance of the composition is significantly increased.

The water resistance can be assessed by the change of the Sun Protection Factor (SPF) upon controlled exposure to water. More details are given in the experimental part.

A further method which has been established for assessing the water resistance is based on the contact angle or change of diameters of a water drop on the surface, see for example Hagens R. et al., “Contact angle measurement—a reliable supportive method for screening water-resistance of ultraviolet-protecting products in vivo”, Internat. J. Cosm. Sci, 2007, 29, 283-291. This method is particularly easy and fast to be performed. More details are given in the experimental part.

Hence, in a further aspect, the present invention relates to the use of a cellulose, which is a C₁₋₆-alkylcellulose or a hydroxy-C₂₋₆-alkyl (C₁₆-alkyl)cellulose or carboxy C₁₋₆-alkyl (C₁₋₆-alkyl)cellulose;
in combination with a mixture of branched and linear saturated C15-C19 alkanes, and an ester of a fatty acid and dextrin;
for increasing the water resistance of a cosmetic composition comprising at least a UV-filter;
   wherein the amount of branched saturated C15-C19 alkane in said mixture of branched and linear saturated C15-C19 alkanes is more than 80% by weight, preferably more that 90% by weight, most preferred more than 92% by weight.

The definitions and preferences of the ingredients have already been described above in great details.

### EXAMPLES

The following examples are provided to further illustrate the compositions and effects of the present invention. These examples are illustrative only and are not intended to limit the scope of the invention in any way.

The cosmetic composition as outlined in table 1 or table 2 have been prepared according to standard methods in the art.

Water Resistance (Change of SPF after Exposure to Water)

1.3 mg/cm² of the respective compositions as outlined in table 1 were applied to 3 PMMA plates (moulded HD6) and spread to yield a homogeneous layer on the plates. Then the plates were dried at room temperature for 15 min. Afterwards the initial in vitro SPF (Sun Protection Factor) (SPFᵢₙᵢₜₐₗ) were determined with a Labsphere UV 2000S with 9 measurement points per plate. Then the plates were immersed into a flask filled with 4 l water (bi-distilled) for 20 min while the water was stirred with a paddle agitator at 150 min⁻¹ at a water temperature of 30° C. (The plates were attached at the edge of the flask with a clothespin, such that the side covered with the composition was directed into the flask). Afterwards the plates were dried at 40° C. for 30 min. The immersion/drying procedure was repeated once. After final drying the plates were cooled to room temperature and the in-vitro SPF was measured as SPF_{immersed} and the water resistance (WR) was calculated as

$$WR = [ ( {SPF}_{immersed} / {SPF}_{initial} ) ]$$

The results are given in table 1.

TABLE 1
Cosmetic composition and its water resistance (SPF) (all ingredients in % by weight)

| Ingredient | INCI name | Ref. 1 | Ref. 2 | Ref. 3 | 1 |
|---|---|---|---|---|---|
| TEGO CARE PBS 6 MB | POLYGLYCERYL-6 STEARATE; | 4 | 4 | 4 | 4 |
| | POLYGLYCERYL-6 BEHENATE; | | | | |
| Imwitor 372 P | GLYCERYL STEARATE CITRATE; | 1 | 1 | 1 | 1 |
| TEGIN ® M Pellets MB | GLYCERYL STEARATE; | 0.5 | 0.5 | 0.5 | 0.5 |
| Lanette ® 16 | CETYL ALCOHOL; | 1 | 1 | 1 | 1 |
| Antaron ® ECo gel | ETHYLCELLULOSE; DIISOPROPYL ADIPATE; | — | 5 | — | 5 |
| Dextrin palmitate¹ | DEXTRIN PALMITATE; | — | — | 2 | 2 |
| Emogreen ™ L19 | C15-19 ALKANE | 5 | 5 | 3 | 3 |
| DUB DIPA | DIISOPROPYL ADIPATE; | 5 | — | 5 | — |
| PARSOL ® EHT | ETHYLHEXYL TRIAZONE; | 4 | 4 | 4 | 4 |
| PARSOL ® 1789 | BUTYL METHOXYDIBENZOYLMETHANE; | 4.7 | 4.7 | 4.7 | 4.7 |
| PARSOL ® Shield | BIS-ETHYLHEXYLOXYPHENOL | 3.8 | 3.8 | 3.8 | 3.8 |
| | METHOXYPHENYL TRIAZINE; | | | | |
| Eldew SL-205 | ISOPROPYL LAUROYL SARCOSINATE; | 3 | 3 | 3 | 3 |
| Tegosoft XC MB | PHENOXYETHYL CAPRYLATE; | 5 | 5 | 5 | 5 |
| Finsolv ® TN | C12-15 ALKYL BENZOATE; | 5 | 5 | 5 | 5 |
| DUB DIS | DIISOPROPYL SEBACATE; | 3 | 3 | 3 | 3 |
| PARSOL ® EHS | ETHYLHEXYL SALICYLATE; | 5 | 5 | 5 | 5 |
| Water Dem | AQUA; | 38.07 | 38.07 | 38.07 | 38.07 |
| Dermofeel ® PA-3 | SODIUM PHYTATE; AQUA; ALCOHOL; | 0.2 | 0.2 | 0.2 | 0.2 |
| VALVANCE ® Touch 210 | SILICA; | 1 | 1 | 1 | 1 |
| Dermosoft ® Octiol | CAPRYLYL GLYCOL; | 0.5 | 0.5 | 0.5 | 0.5 |
| PENTAVITIN ® | SACCHARIDE ISOMERATE; AQUA; CITRIC | 1 | 1 | 1 | 1 |
| | ACID; SODIUM CITRATE; | | | | |
| TILAMAR ® PDO with | PROPANEDIOL; | 2 | 2 | 2 | 2 |
| NØØVISTA ™ | | | | | |
| Cosphaderm ® Pentiol | PENTYLENE GLYCOL; | 4 | 4 | 4 | 4 |
| natural | | | | | |
| PARSOL ® HS | PHENYLBENZIMIDAZOLE SULFONIC ACID; | 1.5 | 1.5 | 1.5 | 1.5 |
| Sodium Hydroxide Pellets | SODIUM HYDROXIDE; | 0.23 | 0.23 | 0.23 | 0.23 |
| Emprove | | | | | |
| Sucrathix VX | MICROCRYSTALLINE CELLULOSE; | 1.5 | 1.5 | 1.5 | 1.5 |
| | CELLULOSE GUM; XANTHAN GUM; | | | | |
| SPFᵢₙᵢₜₐₗ | | 79.4 | 54 | 79.5 | 58.6 |
| SPF_{immersed} | | 9.2 | 14.1 | 41.5 | 50 |
| WR [%] | | 11.6 | 26.1 | 52.2 | 85.3 |

¹Dextrin Palmitate: Mₙ = 11′300-11′500 Da, determined by SEC/GPC

The results of table 1 show that a cosmetic composition comprising different UV filters (Ref.1) has a relative low water resistance of the SPF. When said composition further comprises a specific required cellulose (Ref.2) the water resistance is increased. This increase is less than the increase if said composition comprises an ester of dextrin and a fatty acid (Ref.3). The composition which comprises next to a mixture of branched and linear saturated C15-C19 alkanes also an ester of a fatty acid and dextrin and a specific required cellulose, the water resistance is strongly improved (1 vs Ref.1 vs Ref.2 vs Ref.3).

Water Resistance (Change of Drop Size on Surface after Exposure to Water)

1.2 mg/cm² of the respective compositions as outlined in table 2 were applied to 3 PMMA plates (Schönberg, 5 μm) and spread to yield a homogeneous layer on the plates. Then the plates were dried at room temperature for 5 min. Afterwards a droplet of distilled water of 0.1 ml volume is applied in the middle of the plate. After 1 minute of exposure of the surface to water in said droplet, a photographic picture is taken and the diameter of the droplet is measured. The average of diameter of the drop resulting from the 3 plates per composition is given in table 2 shown in table 2 (Drop Size) as well as the standard deviation (STD) determined from the individual measurements.

The smaller the diameter of the water droplet is, the higher the water resistance of a cosmetic formulation is.

The results of table 2 confirm the findings of table 1. The results of table 2 show that a cosmetic composition comprising different UV filters (Ref.4) has a relatively large diameter and, hence, a low water resistance. When said composition further comprises an ester of dextrin and a fatty acid (Ref.5) the diameter of the droplet is significantly smaller and, hence, the water resistance is increased. This is also the case if said a specific required cellulose (Ref.6) is added to said composition (Ref.4) and, hence, the water resistance is also increased. The composition which comprises next to a mixture of branched and linear saturated C15-C19 alkanes also an ester of a fatty acid and dextrin and a specific required cellulose, the water resistance is strongly improved (2 vs Ref.4 vs Ref.5 vs Ref.6) which is seen by the strong decrease of the diameter of the droplet on the surface of the composition.

TABLE 2
Cosmetic composition and its water resistance (Drop size) (all ingredients in % by weight)

| Ingredient | INCI name | Ref. 4 | Ref. 5 | Ref. 6 | 2 |
|---|---|---|---|---|---|
| Uvinul ® A Plus | DIETHYLAMINO HYDROXYBENZOYL HEXYL | 3.00 | 3.00 | 3.00 | 3.00 |
| | BENZOATE; | | | | |
| PARSOL ® MCX | ETHYLHEXYL METHOXYCINNAMATE; | 5.00 | 5.00 | 5.00 | 5.00 |
| PARSOL ® 340 | OCTOCRYLENE; | 3.50 | 3.50 | 3.50 | 3.50 |
| PARSOL ® SLX | POLYSILICONE-15; | 1.00 | 1.00 | 1.00 | 1.00 |
| UVASORB ® HEB | DIETHYLHEXYL BUTAMIDO TRIAZONE; | 1.00 | 1.00 | 1.00 | 1.00 |
| TEGIN ® M Pellets MB | GLYCERYL STEARATE; | 0.50 | 0.50 | 0.50 | 0.50 |
| Lanette ® 16 | CETYL ALCOHOL; | 1.00 | 1.00 | 1.00 | 1.00 |
| Finsolv ® TN | C12-15 ALKYL BENZOATE; | 5.00 | 5.00 | 5.00 | 5.00 |
| Imwitor 372 P | GLYCERYL STEARATE CITRATE; | 1.00 | 1.00 | 1.00 | 1.00 |
| Dermosoft ® Octiol | CAPRYLYL GLYCOL; | 0.50 | 0.50 | 0.50 | 0.50 |
| Tegosoft XC MB | PHENOXYETHYL CAPRYLATE; | 5.00 | 5.00 | 5.00 | 5.00 |
| Eldew SL-205 | ISOPROPYL LAUROYL SARCOSINATE; | 3.00 | 3.00 | 3.00 | 3.00 |
| TEGO CARE PBS 6 MB | POLYGLYCERYL-6 STEARATE; | 4.00 | 4.00 | 4.00 | 4.00 |
| | POLYGLYCERYL-6 BEHENATE; | | | | |
| Antaron ® ECo gel | ETHYLCELLULOSE; DIISOPROPYL ADIPATE; | — | — | 5.00 | 5.00 |
| Dextrin palmitate¹ | DEXTRIN PALMITATE; | — | 2.00 | — | 2.00 |
| EMOGREEN L19 | C15-19 ALKANE; | 5.00 | 3.00 | 5.00 | 3.00− |
| DUB DIPA | DIISOPROPYL ADIPATE; | 5.00 | 5.0− | — | — |
| PARSOL ® TX | TITANIUM DIOXIDE; SILICA; DIMETHICONE; | 1.00 | 1.00 | 1.00 | 1.00 |
| Water Dem | AQUA; | 42.80 | 42.80 | 42.80 | 42.80 |
| Dermofeel ® PA-3 | SODIUM PHYTATE; AQUA; ALCOHOL; | 0.20 | 0.20 | 0.20 | 0.20 |
| Cosphaderm ® Pentiol | PENTYLENE GLYCOL; | 4.00 | 4.00 | 4.00 | 4.00 |
| natural | | | | | |
| TILAMAR ® PDO with | PROPANEDIOL; | 2.00 | 2.00 | 2.00 | 2.00 |
| NØØVISTA ™ | | | | | |
| Sucrathix VX | MICROCRYSTALLINE CELLULOSE; | 1.50 | 1.50 | 1.50 | 1.50 |
| | CELLULOSE GUM; XANTHAN GUM; | | | | |
| PARSOL ® Max | METHYLENE BIS-BENZOTRIAZOLYL | 3.00 | 3.00 | 3.00 | 3.00 |
| | TETRAMETHYLBUTYLPHENOL; AQUA; DECYL | | | | |
| | GLUCOSIDE; PROPYLENE GLYCOL; XANTHAN | | | | |
| | GUM; | | | | |
| PENTAVITIN ® | SACCHARIDE ISOMERATE; AQUA; CITRIC | 1.00 | 1.00 | 1.00 | 1.00 |
| | ACID; SODIUM CITRATE; | | | | |
| VALVANCE ® Touch 210 | SILICA; | 1.00 | 1.00 | 1.00 | 1.00 |
| Drop Size [mm] | | 27.1 | 20.8 | 19.7 | 16.6 |
| STD [mm] | | 0.9 | 1.7 | 1.3 | 1.2 |

¹Dextrin Palmitate: Mₙ = 11′300-11′500 Da, determined by SEC/GPC.

1. A cosmetic composition comprising
a UV-filter
a mixture of branched and linear saturated C15-C19 alkanes; and
an ester of a fatty acid and dextrin; and
a cellulose which is a C1-6-alkylcellulose or a hydroxy-C2-6-alkyl (C1-6-alkyl)cellulose or carboxy C1-6-alkyl (C1-6-alkyl)cellulose
wherein the amount of branched saturated C15-C19 alkane in said mixture of branched and linear saturated C15-C19 alkanes is more than 80% by weight, preferably more that 90% by weight, most preferred more than 92% by weight.

2. The cosmetic composition according to claim 1, wherein the amount of linear saturated C15-C19 alkanes in said mixture of branched and linear saturated C15-C19 alkanes is less than 10% by weight, preferably less than 8% by weight, most preferred more than 5% by weight.

3. The cosmetic composition according to claim 1, wherein the amount of branched saturated C18 alkane is more than 50% by weight, preferably more than 60% by weight, even more preferably more than 70% by weight, relative to the weight of said mixture of branched and linear saturated C15-C19 alkanes.

4. The cosmetic composition according to claim 1, wherein the fatty acid of said ester of a fatty acid and dextrin is a C14-C18 fatty acid, particularly a linear C14-C18 fatty acid, most preferably palmitic acid.

5. The cosmetic composition according to claim 1, wherein the dextrin of said ester of a fatty acid and dextrin has an average degree of glycopolymerization of between 3 and 20, particularly between 8 and 16.

6. The cosmetic composition according to claim 1, wherein said ester of a fatty acid and dextrin has an average number of esterified hydroxyl groups of more than 2.5, preferably between 2.5 and 3.5, more preferably between 2.5 and 3.4, most preferably between 2.5 and 3.2, per glucose unit.

7. The cosmetic composition according to claim 1, wherein said ester of a fatty acid and dextrin has a molecular weight Mₙ of between 8,000 and 16,000 Da, preferably between 9,000 and 13,000 Da, more preferably between 10,000 and 11,500 Da.

8. The cosmetic composition according to claim 1, wherein the UV filter is selected from the group consisting of ethylhexyl triazone, avobenzone, bis-ethylhexyloxyphenol methoxyphenyl triazine, 2-ethylhexyl salicylate, phenylbenzimidazole sulfonic acid, ethylhexyl methoxycinnamate, octocrylene, polysilicone-15, iscotrizinol, titanium dioxide, and methylene bis-benzotriazolyl tetramethylbutylphenol

9. The cosmetic composition according to claim 1, wherein the cellulose is a selected from the group consisting of methylcellulose, ethylcellulose, propylcellulose, hydroxymethyl cellulose, hydroxyethyl cellulose, hydroxypropyl cellulose, hydroxyethyl methylcellulose, hydroxypropyl methylcellulose, carboxymethyl cellulose, carboxymethyl methylcellulose and carboxymethyl ethylcellulose particularly an ethylcellulose.

10. The cosmetic composition according to claim 1, wherein the amount of cellulose is in the range of between 0.01 and 4.0% by weight, particularly between 0.1 and 3.0% by weight, preferably between 0.3 and 2.5% by weight, based on the weight of the cosmetic composition.

11. The cosmetic composition according to claim 1, wherein the amount of the mixture of branched and linear saturated C15-C19 alkanes is in the range of between 0.5 and 12.0% by weight, particularly between 1.0 and 10.0% by weight, more preferably between 1.5 and 8.0% by weight, preferably between 2.0 and 6.0% by weight, based on the weight of the cosmetic composition.

12. The cosmetic composition according to claim 1, wherein the amount of the ester of a fatty acid and dextrin is in the range of between 0.3 and 8.0% by weight, particularly between 0.6 and 7.0% by weight, more preferably between 1.0 and 6.0% by weight, particularly between 1.5 and 5.0% by weight, based on the weight of the cosmetic composition.

13. The cosmetic composition according to claim 1, wherein the composition comprises water and is in the form of an emulsion.

14. The cosmetic composition according to claim 1, wherein the composition has a Sun Protection Factor (SPF) of 10 or higher, preferably of 20 or higher, more preferred of 30 or higher.

15. Use of a cellulose, which is a C₁₋₆-alkylcellulose or a hydroxy-C₂₋₆-alkyl (C₁₆-alkyl)cellulose or carboxy C₁₋₆-alkyl (C₁₋₆-alkyl)cellulose;
in combination;
for increasing the water resistance of a cosmetic composition comprising at least a UV-filter;
wherein the amount of branched saturated C15-C19 alkane in said mixture of branched and linear saturated C15-C19 alkanes is more than 80% by weight, preferably more that 90% by weight, most preferred more than 92% by weight.
